(19) United States
(12) Patent Application Publication
(10) Pub. No.: US 2002/0000539 A1
(43) Pub. Date: Jan. 3, 2002

(54) Bulk nickel catalysts and processes for the production of syngas

(76) Inventors: Anne M. Gaffney, West Chester, PA (US); Robert A. Oswald, Milton, DE (US); Roger Song, Wilmington, DE (US); Juan C. Figueroa, Wilmington, DE (US)

Correspondence Address: Joanna K. Payne, CONOCO INC., 1000 South Pine 2635 RW, P.O. Box 1267, Ponca City, OK, 74602-1267, US

(21) Appl. No.: 09/753,103

(22) Filed: Jan. 2, 2001

Related U.S. Application Data
(60) Provisional application No. 60/174,889, filed on Jan. 7, 2000.

(51) Int. Cl. B01J 23/00; C10K 1/00; C07C 1/02; C01B 3/26; C01B 31/18; C01B 31/24

(52) U.S. Cl. 252/373; 502/337; 423/651; 423/418.2

(57) ABSTRACT

A method is disclosed for the catalytic conversion of light hydrocarbons to synthesis gas. The method involves the contacting of a feed stream comprising the hydrocarbon feedstock and an O₂-containing gas with a catalyst in a reaction zone maintained at conversion-promoting conditions effective to produce an effluent stream comprising carbon monoxide and hydrogen. The preferred catalysts of the invention comprise bulk nickel monoliths that have been activated by heating in a reducing environment. The preferred catalysts convert hydrocarbons to syngas primarily by a predominantly partial oxidation reaction and retain a high level of activity and selectivity to carbon monoxide and hydrogen under conditions of elevated pressure, high gas space velocity and high temperature in a short contact time reactor.

### CROSS-REFERENCE TO RELATED APPLICATIONS

This application claims the benefit under 35 U.S.C. § 119(e) of U.S. Provisional Patent Application No. 60/174,889 filed Jan. 7, 2000.

### BACKGROUND OF THE INVENTION

1. Field of the Invention

The present invention generally relates to processes for catalytically converting light hydrocarbons (e.g., natural gas) to a product containing carbon monoxide and hydrogen by employing a bulk nickel metal catalyst. More particularly, the invention relates to reduced bulk Ni monolithic catalysts capable of catalyzing the net partial oxidation of methane or other light hydrocarbons, and to synthesis gas generation processes employing such catalysts.

2. Description of Related Art

Large quantities of methane, the main component of natural gas, are available in many areas of the world, and natural gas is predicted to outlast oil reserves by a significant margin. However, most natural gas is situated in areas that are geographically remote from population and industrial centers. The costs of compression, transportation, and storage make its use economically unattractive.

To improve the economics of natural gas use, much research has focused on methane as a starting material for the production of higher hydrocarbons and hydrocarbon liquids. The conversion of methane to hydrocarbons is typically carried out in two steps. In the first step, methane is reformed with water to produce carbon monoxide and hydrogen (i.e., synthesis gas or syngas). In a second step, the syngas is converted to hydrocarbons, for example, using the Fischer-Tropsch process to provide fuels that boil in the middle distillate range, such as kerosene and diesel fuel, and hydrocarbon waxes. Present day industrial use of methane as a chemical feedstock typically proceeds by the initial conversion of methane to carbon monoxide and hydrogen by either steam reforming, which is the most widely used process, or by dry reforming. Steam reforming proceeds according to Equation 1.

CH₄+H₂⇄CO+3H₂ (1)

Although steam reforming has been practiced for over five decades, efforts to improve the energy efficiency and reduce the capital investment required for this technology continue.

The partial oxidation of hydrocarbons, e.g., natural gas or methane is another process that has been employed to produce syngas. While currently limited as an industrial process, partial oxidation has recently attracted much attention due to significant inherent advantages, such as the fact that significant heat is released during the process, in contrast to the steam reforming processes, which are endothermic. Partial oxidation of methane proceeds exothermically according to the following reaction stoichiometry:

CH₄+½O₂⇄CO+2H₂ (2)

In the catalytic partial oxidation processes, natural gas is mixed with air, oxygen or oxygen-enriched air, and is introduced to a catalyst at elevated temperature and pressure. The partial oxidation of methane yields a syngas mixture with a H₂:CO ratio of 2:1, as shown in Equation 2. This ratio is more useful than the H₂:CO ratio from steam reforming for the downstream conversion of the syngas to chemicals such as methanol and to fuels. Furthermore, oxidation reactions are typically much faster than reforming reactions. This makes possible the use of much smaller reactors for catalytic partial oxidation processes. The syngas in turn may be converted to hydrocarbon products, for example, fuels boiling in the middle distillate range, such as kerosene and diesel fuel, and hydrocarbon waxes by processes such as the Fischer-Tropsch synthesis.

The selectivities of catalytic partial oxidation to the desired products, carbon monoxide and hydrogen, are controlled by several factors, but one of the most important of these factors is the choice of catalyst composition. Difficulties have arisen in the prior art in making such a choice economical. Typically, catalyst compositions have included precious metals and/or rare earths. The large volumes of expensive catalysts needed by the existing catalytic partial oxidation processes have placed these processes generally outside the limits of economic justification.

A number of process regimes have been described in the literature for the production of syngas via catalyzed partial oxidation reactions. The noble metals, which typically serve as the best catalysts for the partial oxidation of methane, are scarce and expensive. The more widely used, less expensive, catalysts have the disadvantage of promoting coke formation on the catalyst during the reaction, which results in loss of catalytic activity. Moreover, in order to obtain acceptable levels of conversion of gaseous hydrocarbon feedstock to CO and H₂ it is typically necessary to operate the reactor at a relatively low flow rate, or space velocity, using a large quantity of catalyst.

For successful operation at commercial scale, however, the catalytic partial oxidation process must be able to achieve a high conversion of the methane feedstock at high gas hourly space velocities, and the selectivity of the process to the desired products of carbon monoxide and hydrogen must be high. Such high conversion and selectivity must be achieved without detrimental effects to the catalyst, such as the formation of carbon deposits (“coke”) on the catalyst, which severely reduces catalyst performance. Accordingly, substantial effort has been devoted in the art to the development of economical catalysts allowing commercial performance without coke formation. Not only is the choice of the catalyst's chemical composition important, the physical structure of the catalyst and catalyst support structures must possess mechanical strength and porosity, in order to function under operating conditions of high pressure and high flow rate of the reactant and product gasses. Another object of continuing efforts in this field is to develop stronger, more porous catalyst supports.

Of the methods that employ nickel-containing catalysts for oxidative conversion of methane to syngas, typically the nickel is supported by alumina or some other type of ceramic support. For example, V. R. Choudhary et al. (J. Catal., Vol. 172, pages 281-293, 1997) disclose the partial oxidation of methane to syngas at contact times of 4.8 ms (at STP) over supported nickel catalysts at 700 and 800° C. The catalysts were prepared by depositing NiO-MgO on different commercial low surface area porous catalyst carriers consisting of refractory compounds such as SiO₂, Al₂O₃, SiC, ZrO₂ and HfO₂. Catalysts were also prepared by depositing NiO on the catalyst carriers with different alkaline and rare earth oxides such as MgO, CaO, SrO, BaO, Sm₂O₃ and Yb₂O₃.

U.S. Pat. No. 5,149,464 discloses a method for selectively converting methane to syngas at 650° C. to 950° C. by contacting the methane/oxygen mixture with a solid catalyst, which is either:

(a) a catalyst of the formula MₓM′_{y}O_{z} where: M is at least one element selected from Mg, B, Al, Ln, Ga, Si, Ti, Zr and Hf; Ln is at least one member of lanthanum and the lanthanide series of elements, M′ is a d-block transition metal, and each of the ratios x/z and y/z and (x+y)/z is independently from 0.1 to 8. Alternatively, the catalyst is (b) an oxide of a d-block transition metal; or (c) a d-block transition metal on a refractory support; or (d) a catalyst formed by heating a) or b) under the conditions of the reaction or under non-oxidizing conditions. The d-block transition metals are selected from those having atomic number 21 to 29, 40 to 47 and 72 to 79, the metals Sc, Ti, Va, Cr, Mn, Fe, Co, Ni, Cu, Zr, Nb, Mo, Tc, Ru, Rh, Pa, Ag, Hf, Ta, W, Re, Os, Ir, Pt and Au.

U.S. Pat. No. 5,500,149 describes a Ni/Al₂O₃ catalyst that catalyzes the reaction CO₂+CH₄→2CO+2H₂, and demonstrates how reaction conditions can affect the product yield. The partial oxidation of methane to synthesis gas using various transition metal catalysts under a range of conditions has been described by Vernon, D. F. et al. (Catalysis Letters 6:181-186 (1990)). European Pat. App. Pub. No. 640561 discloses a catalyst for the catalytic partial oxidation of hydrocarbons comprising a Group VIII metal on a refractory oxide having at least two cations. Multimonolith combustors are discussed by M. F. M. Zwinkels, et al. in a chapter entitled “Catalytic Fuel Combustion in Honeycomb Monolith Reactors” in Structured Catalysts and Reactors (A. Cybulski et al., eds. 1998. Marcel Dekker, Inc.,Ch. 6, pp. 149-177.)

European Patent No. EP 303,438 describes a catalytic partial oxidation process for converting a hydrocarbon feedstock to synthesis gas using steam in addition to oxygen. The exemplary reaction is catalyzed by a monolith of Pt-Pd on an alumina/cordierite support. Certain catalyst disks of dense wire mesh, such as high temperature alloys or platinum mesh are also described. Optionally, the wire mesh may be coated with certain metals or metal oxides having catalytic activity for the oxidation reaction.

M. D. Pawson et al. disclosed that Ni gauze is relatively inert as a catalyst for oxidation of methane in air at temperatures of about 1000° C., while Pt and Pt-Rh are catalytically active (“An LIF Study of Methane Oxidation over Noble Metal Gauze Catalysts” Abstracts 1999 Meeting Dallas, Texas Assoc. Indust. Chem. Eng., p. 289b). Those investigators also showed that 40-mesh nickel gauze did not ignite and there was no conversion of methane under methane partial oxidation conditions. It was concluded that bulk Ni metal is inert towards the conversion of methane to syngas (Davis, M., et al. Combustion and Flame 123: 159-174 (2000)).

U.S. Pat. Nos. 3,957,682 and 4,083,799 (assigned to Texaco, Inc.) disclose an iconel metal screen consisting of about 50-95% nickel that is a methane steam reforming catalyst. In these processes the Ni catalyst is initially activated by heating in an oxygen-containing gas. Similarly, U.S. Pat. No. 5,112,527 (assigned to Amoco Corporation) also describe Ni as a reforming catalyst in the presence of steam, a gaseous lower alkane and air and in combination with a Group VIII metal having partial oxidation activity.

One disadvantage of many of the existing catalytic hydrocarbon conversion methods is the need, in many cases, to include steam in the feed mixture to suppress coke formation on the catalyst. Another drawback of some of the existing processes is that the catalysts that are employed often result in the production of significant quantities of carbon dioxide, steam, and C₂+hydrocarbons. Also, in order to operate at very high flow rates, high pressure and smaller catalyst beds of the smaller, short contact time reactors employed for partial oxidation processes it is necessary to employ a very porous, highly active and mechanically strong catalyst. None of the existing catalytic partial oxidation processes are capable of providing sufficiently high conversion of reactant gas and high selectivity of CO and H₂ reaction products without employing rare and costly catalysts. Accordingly, there is a continuing need for better, more economical processes and catalysts for the catalytic partial oxidation of hydrocarbons, particularly methane, or methane containing feeds, in which the catalyst retains a high level of activity and selectivity to carbon monoxide and hydrogen under conditions of high gas space velocity, elevated pressure and high temperature.

### SUMMARY OF THE INVENTION

Processes for preparing synthesis gas using activated bulk Ni structures for converting a gaseous hydrocarbon having a low boiling point (e.g. C₁-C₅ hydrocarbons, particularly methane, or methane containing feeds) are provided. The activated bulk or monolithic metallic nickel catalysts, and their method of making, are also described. One advantage of the preferred catalysts is that they retain a high level of activity and selectivity to carbon monoxide and hydrogen products and support high gas space velocities under conditions of elevated pressure and high temperature, in contract to conventional nickel-containing catalysts which often fail under those conditions. The reaction stoichiometry favors the catalytic partial oxidation reaction as the primary reaction catalyzed by the preferred catalysts. Another advantage provided by the preferred new catalysts and processes is that they are economically feasible for use under commercial-scale conditions. The new syngas production processes are particularly useful for converting gas from naturally occurring reserves of methane which contain carbon dioxide. In accordance with certain embodiments the bulk Ni catalyst is in any of various three-dimensional forms such as monoliths, disks or pieces consisting of gauzes, foams, perforated foils, spirally wound foils (“spirals”), expanded Ni metal, and the like, the structure being sufficiently porous, permeable or transparent to permit a gas/catalyst contact time of no more than about 10 milliseconds when the catalyst is employed in a millisecond contact time syngas production reactor. When activated for catalyzing the partial oxidation of a light hydrocarbon, such as methane, the bulk Ni metal is preferably in its reduced state, i.e., Ni⁰. Activation may be accomplished by heating in a reducing atmosphere prior to commencing contact with the hydrocarbon feedstock and oxygen containing gas. This activation pre-treatment can be done either in situ in the reactor or outside of the reactor, and maintained in a reduced state until use.

Also provided by the present invention are methods of partially oxidizing a 1-5 carbon-containing gaseous hydrocarbon, such as methane, to form a product gas mixture comprising CO and H₂. The processes include maintaining the catalyst and the reactant gas mixture at conversion promoting conditions of temperature, pressure, contact time, and hydrocarbon and O₂ concentration and atomic ratio. Preferably the method includes maintaining the catalyst at a temperature of about 600-1,200° C. during contact. In some embodiments the temperature is maintained at about 700-1,100° C. In some embodiments of the methods the reactant gases are maintained at a pressure of about 100-12,500 kPa during the contacting, and in some of the more preferred embodiments the pressure is maintained at about 130-10,000 kPa.

Certain embodiments of the methods of converting hydrocarbons to CO and H₂ comprise mixing a methane-containing feedstock and an O₂-containing feedstock to provide a reactant gas mixture feedstock having a carbon:oxygen ratio of about 1.25:1 to about 3.3:1. In some of these embodiments, the mixing step is such that it yields a reactant gas mixture feed having a carbon:oxygen ratio of about 1.3:1 to about 2.2:1, or about 1.5:1 to about 2.2:1. In some of the most preferred embodiments the mixing step provides a reactant gas mixture feed having a carbon:oxygen ratio of about 2:1.

In some embodiments of the methods the said oxygen-containing gas that is mixed with the hydrocarbon comprises steam or CO₂, or a mixture of both. In some embodiments of the methods the C₁-C₅ hydrocarbon comprises at least about 50% methane by volume, and in some of the preferred embodiments the C₁-C₅ hydrocarbon comprises at least about 80% methane by volume.

Certain embodiments of the processes for converting light hydrocarbons to syngas comprise preheating the reactant gas mixture prior to contacting the catalyst. For example, in some cases the reactant gases may be preheated up to about 600° C. to facilitate ignition. Some embodiments of the processes comprise passing the reactant gas mixture over the catalyst at a space velocity of about 20,000 to about 100,000,000 normal liters of gas per kilogram of catalyst per hour (NL/kg/h). In certain of these embodiments, the gas mixture is passed over the catalyst at a space velocity of about 50,000 to about 50,000,000 NL/kg/h. In preferred embodiments the selectivity of the process for CO and H₂ products is such that the molar ratio of H₂:CO in the product gas mixture is about 2:1, as in Equation (2), above, suitable for feeding directly into a Fischer-Tropsch process.

These and other embodiments, features and advantages of the present invention will become apparent with reference to the following description.

### DETAILED DESCRIPTION OF PREFERRED EMBODIMENTS

A bulk or monolithic nickel metal catalyst capable of converting C₁-C₅ hydrocarbons to CO and H₂ is prepared as described in the following examples and may have any of various 3-D forms such as gauzes, foams, foils, spirals, expanded Ni metal, and the like. Preferably, however, the bulk Ni catalyst is prepared from an expanded Ni metal sheet.

Contrary to the general consensus that bulk Ni is not very useful for catalyzing the oxidation of methane, the present inventors now demonstrate that by properly activating the new bulk Ni catalysts in a reducing environment, an active, selective and productive syngas catalyst is produced. Preferred bulk nickel catalyst structures prepared as described in the following examples are highly active catalysts with sufficient mechanical strength to withstand high pressures and temperatures and permit a high flow rate of reactant and product gases when employed on-stream in a short contact time reactor for synthesis gas production. Any suitable reaction regime may be applied in order to contact the reactants with the catalyst. One suitable regime is a fixed bed reaction regime, in which the catalyst is retained within a reaction zone in a fixed arrangement. The bulk Ni catalyst is employed in the fixed bed regime, retained using fixed bed reaction techniques that are well known and have been described in the literature.

### EXAMPLE 1

### Expanded Ni Metal

Nineteen (19) disks 12 mm in diameter were prepared from a sheet of expanded Ni metal obtained from Exmet Corporation of Naugatuck, Conn. Preferably the Ni content is about 100%. The Exmet specification for the expanded Ni metal was 4 Ni X-4/0. The thickness of each disk was 0.004″. The long-way of the diamond (LWD) shape was 2 mm and the short-way of the diamond (SWD) shape was 1 mm. The disks were initially cleaned by the following procedure. The disks were soaked in 50 ml of acetone for 30 minutes, followed by immersion in 20 ml of 20 wt % NaOH at room temperature for 20 minutes. This NaOH solution with the immersed disks was then heated to 80° C. and held for 20 minutes at 80° C. Subsequently, the disks were rinsed with deinonized water until the washing was neutral. The disks were dried in a vacuum oven at 110° C. for 2 hours prior to charging to the reactor for testing. Suitable expanded Ni metal sheets from which the disks may be formed are listed in Table 1, although any other expanded Ni metal configuration may be employed as long as the pressure drop of the final catalyst is acceptable for the particular syngas production system. The 19 expanded Ni metal disks, stacked together, were charged to the reactor for testing, as described below under “Test Procedure,” employing a short contact time syngas synthesis reactor. After six attempted reaction ignitions, whereby the feed composition of 60% CH₄, 30% O₂ and 10% N₂ is passed over the disks at temperatures ranging up to 800° C., there was no ignition and no methane conversion.

TABLE 1

| | Mesh Dimensions | Mesh Dimensions | Thickness of | Strand |
|---|---|---|---|---|
| Mesh | (from center to center of joints) | (from center to center of joints) | Original Material | Width |
| Designation | Long Way of the Diamond | Short Way of the Diamond | (min./max.) | (min./max.) |
| (size) | (inches) | (min./max.) (inches) | (inches) | (inches) |
| 3/16 | .506 | .200-.279 | .010/.040 | .015/.070 |
| 1 | .405 | .200-.235 | .003/.025 | .007/.055 |
| 1 HX | .405 | .214-.240 | .010/.040 | .015/.060 |
| 1/0 | .280 | .100-.150 | .003/.025 | .007/.055 |
| 1/0 HX | .278 | .166-.200 | .010/.035 | .015/.050 |
| 1/23 | .236 | .126-.143 | .003/.035 | .005/.050 |
| 1/22 | .2284 | .107-.120 | .005/.030 | .010/.040 |
| 3/32 | .215 | .107-.143 | .002/.030 | .005/.045 |
| 2/0 | .187 | .077-.091 | .002/.020 | .007/.035 |
| 2/0 HX | .190 | .118-.143 | .005/.030 | .010/.045 |
| 2/0 E | .187 | .056-.071 | .002/.010 | .007/.035 |
| 2/1 | .180 | .091-.111 | .003/.026 | .005/.026 |
| 3/2 HX | .1575 | .102-.115 | .005/.026 | .010/.040 |
| 3/1 | .140 | .080-.091 | .002/.024 | .004/.026 |
| 3/0 | .125 | .053-.071 | .002/.015 | .004/.020 |
| 3/0 HX | .125 | .077-.083 | .005/.020 | .007/.025 |
| 4/4 HX | .105 | .071-.074 | .005/.026 | .005/.026 |
| 4/3 | .100 | .050-.059 | .002/.015 | .004/.020 |
| 4/3 HX | .100 | .063-.069 | .004/.018 | .005/.022 |
| 4/2 HX | .093 | .063-.065 | .004/.018 | .005/.022 |
| 4/1 | .080 | .048-.053 | .002/.015 | .004/.020 |
| 4/0 | .077 | .033-.046 | .002/.012 | .004/.020 |
| 5/0 | .050 | .027-.031 | .0021.010 | .004/.012 |
| 6/0 | .031 | .021-.024 | .002/.007 | .004/.010 |

Source: Exmet Corporation, Naugatuck, Conn.

### EXAMPLE 2

### Activated Expanded Ni Metal

Bulk Ni disks prepared as described in Example 1 were activated in a reducing environment by passing 100 cc/min H₂ over the disks at 800° C. for 4 hours inside the reactor. Alternatively, the disks may be activated outside of the reactor and maintained in reduced condition until use. The H₂ stream may be diluted with an inert gas such as N₂, if desired. Subsequent to this treatment, 19 activated disks were tested in the short contact time partial oxidation reactor, as described in Example 1. In this case, ignition occurred at 350° C. with the feed composition of 60% CH₄, 30% O₂ and 10% N₂. At a run temperature of 1065° C. and a flow rate of 2.5 SLPM, 78% CH₄ conversion and 100% O₂ conversion were observed and 95% CO selectivity and 71% H₂ selectivity were obtained in the product gas mixture. The observed stoichiometry of the reactants and products is consistent with catalytic partial oxidation being the predominant type of reaction taking place (i.e., H₂:CO ratio about 2: 1, as in Equation (2), above). Although expanded nickel metal disks were employed in these representative demonstrations, satisfactory bulk Ni catalysts prepared in any of a variety of other three-dimensional forms such as nickel gauzes, foams, perforated foils, spirals, and the like, would also perform satisfactorily. The observed reaction stoichiometry catalyzed by the bulk Ni catalysts is predominantly the partial oxidation of the hydrocarbon. Variation of the catalyst composition, however, influences the relative contributions of alternate reactions like combustion, steam reforming, CO₂ reforming and water gas shift, which are also present under these reaction conditions, but to a lesser extent than the partial oxidation reaction.

Preferably, the bulk Ni catalyst is prepared from an expanded Ni metal sheet that has been simultaneously slit and stretched by shaped tools which determine the form and number of openings. Strand dimensions (width and thickness), overall thickness of the piece and weight per square inch are controlled variables. The controlled percentage of opening can be extremely light and open, i.e., as high as 90% open area. The expanded metal structure has certain advantages over other open area materials for forming the monolithic catalyst. For example, one square foot of perforated material produces only one square foot of product. For expanded metals, however, there is no waste and one square foot of material results in two or three times and even more of finished product. One alternative to an expanded metal bulk Ni catalyst is one prepared from perforated Ni foil. Perforation processes such as abrasive drilling, laser drilling, electron beam drilling, electric discharge machining, photochemical machining, or another technique familiar to those skilled in the art can be conveniently used to prepare the base structure for forming the bulk catalyst. In producing woven wire or cloth, the process must start with wire, drawn and annealed to the correct diameter. Depending on their rigidity, the intersecting strands are relatively free to move past each other. With the expanded or the perforated metal, however, the strands are integral and the result is a remarkably strong material. A variety of suitable bulk nickel starting materials from which the catalysts can be prepared are commercially available, for example, from Exmet Corporation, Naugatuck, Conn. Alternative 3-D shapes can also be formed using appropriate metal shaping or forming techniques that have been described in the literature. For example, methods of making porous metal foams are described in PCT publication WO 97/31738 (assigned to Astro Met, Inc.). Techniques which enhance the stiffness of the metal foam to better support a large foam structure are preferred. Also, techniques that reduce or eliminate impurities in the metal foam, which hinder the catalytic performance, are desirable.

### Test Procedure

Several schemes for carrying out catalytic partial oxidation (CPOX) of hydrocarbons in a short contact time reactor have been described in the literature. For example, L. D. Schmidt and his colleagues at the University of Minnesota describe a millisecond contact time reactor in U.S. Pat. No. 5,648,582 and in J Catalysis 138, 267-282 (1992) for use in the production of synthesis gas by direct oxidation of methane over a catalyst such as platinum or rhodium. A general description of major considerations involved in operating a reactor using millisecond contact times is given in U.S. Pat. No. 5,654,491. The disclosures of the above-mentioned references are incorporated herein by reference. In the present studies, the above-described catalysts were evaluated in a conventional flow apparatus using a 19 mm O.D.×13 mm I.D. and 12″ long quartz reactor. A ceramic foam of 99% Al₂O₃ (12 mm OD×5 mm of 45 ppi) were placed before and after the catalyst as radiation shields. The inlet radiation shield also aided in uniform distribution of the feed gases. An Inconel sheathed, single point K-type (Chromel/Alumel) thermocouple (TC) was placed axially inside the reactor touching the top (inlet) face of the radiation shield. A high temperature S-Type (Pt/Pt 10% Rh) bare-wire TC was positioned axially touching the bottom face of the catalyst and was used to indicate the reaction temperature. The catalyst and the two radiation shields were sealed tight against the walls of the quartz reactor by wrapping them radially with a high purity (99.5%) alumina paper. A 600 watt band heater set at 90% electrical output was placed around the quartz tube, providing heat to light off the reaction and to preheat the feed gases. For example, in some instances it is desirable to preheat the feed gases up to about 600° C. to ignite the reaction. The bottom of the band heater corresponded to the top of the upper radiation shield.

In addition to the TCs placed above and below the catalyst, the reactor also contained two axially positioned, triple-point TCs, one before and another after the catalyst. These triple-point thermocouples were used to determine the temperature profiles of reactants and products subjected to preheating and quenching, respectively. Preheating was done with the 600 watt band heater and quenching was accomplished with water cooling coils wrapped around the external surface of the lower section of the tubular reactor.

All test runs were done at a reactant gas feed mixture of CH₄:O₂ at a molar ratio of 2:1, and at a pressure of 5 psig (136 kPa). The reactor effluent was analyzed using a gas chromatograph equipped with a thermal conductivity detector. In the present studies, the C, H and O mass balance were all between 98-102%.

### Process of Producing Syngas

A feed stream comprising a light hydrocarbon feedstock, such as methane, and an oxygen-containing gas is contacted with an activated bulk Ni catalyst, prepared as described in Example 2. The activated bulk Ni catalyst is contained in a reaction zone maintained at conversion-promoting conditions effective to produce an effluent stream comprising carbon monoxide and hydrogen. Preferably a millisecond contact time reactor is employed. The hydrocarbon feedstock may be any gaseous hydrocarbon having a low boiling point, such as methane, natural gas, associated gas, or other sources of light hydrocarbons having from 1 to 5 carbon atoms. The hydrocarbon feedstock may be a gas arising from naturally occurring reserves of methane which contain carbon dioxide. Preferably, the feed comprises at least 50% by volume methane, more preferably at least 75% by volume, and most preferably at least 80% by volume methane.

The hydrocarbon feedstock is in the gaseous phase when contacting the catalyst. The hydrocarbon feedstock is contacted with the catalyst as a mixture with an oxygen-containing gas, preferably pure oxygen. The oxygen-containing gas may also comprise steam and/or CO₂ in addition to oxygen. Alternatively, the hydrocarbon feedstock is contacted with the catalyst as a mixture with a gas comprising steam and/or CO₂. It is preferred that the methane-containing feed and the oxygen-containing gas are mixed in such amounts to give a carbon (i.e., carbon in methane) to oxygen (i.e., oxygen) ratio from about 1.25:1 to about 3.3:1, more preferably, from about 1.3:1 to about 2.2:1, and most preferably from about 1.5:1 to about 2.2:1, especially the stoichiometric ratio of 2:1. The process is operated at atmospheric or superatmospheric pressures, the latter being preferred. The pressures may be from about 100 kPa to about 12,500 kPa, preferably from about 130 kPa to about 10,000 kPa. The process is preferably operated at temperatures of from about 600° C. to about 1200° C., preferably from about 700° C. to about 1100° C. The hydrocarbon feedstock and the oxygen-containing gas are preferably pre-heated before contact with the catalyst. The hydrocarbon feedstock and the oxygen-containing gas are passed over the catalyst at any of a variety of space velocities. The gas flow rate is preferably regulated such that the contact time for the portion of reactant gas mixture that contacts the catalyst is no more than about 10 milliseconds and more preferably from about 1 to 5 milliseconds. This ultra short contact time is accomplished by passing the reactant gas mixture over one of the above-described catalysts at a space velocity, stated as normal liters of gas per kilogram of catalyst per hour, of about 20,000 to about 100,000,000 NL/kg/h, preferably about 50,000 to about 50,000,000 NL/kg/h. The product gas mixture emerging from the reactor are, optionally, sampled for analysis of products, including CH₄, O₂, CO, H₂ and CO₂, and then harvested or routed to another application such as a Fischer-Tropsch process.

While the preferred embodiments of the invention have been shown and described, modifications thereof can be made by one skilled in the art without departing from the spirit and teachings of the invention. The embodiments described herein are exemplary only, and are not intended to be limiting. For example, pure methane was employed in the representative test procedures, however, any light hydrocarbon (i.e., C₁-C₅) gaseous feedstock could also serve as a feedstock for the catalytic conversion of light hydrocarbons by the new bulk Ni catalysts. Many variations and modifications of the invention disclosed herein are possible and are within the scope of the invention. For example, the 3-D shapes named by the inventors are only a few of the many workable configurations the new catalysts may assume, provided that the pressure drop associated with on-stream use of the final monolith is not excessive. Accordingly, the scope of protection is not limited by the description set out above, but is only limited by the claims which follow, that scope including all equivalents of the subject matter of the claims. The disclosure of U.S. Provisional Patent Application No. 60/174,889, and the disclosures of all patents and publications cited herein are incorporated by reference.

What is claimed is:

1. A method of catalytically converting a C₁-C₅ hydrocarbon to a product gas mixture comprising CO and H₂, the method comprising:
in a millisecond contact time reactor, contacting a reactant gas mixture comprising said hydrocarbon and O₂ with a catalytically effective amount of a metallic nickel monolith catalyst, said nickel being in its reduced state (Ni⁰) and said monolith catalyst having a structure that is sufficiently transparent to allow reactant and/or product gases to pass through said monolith such that a portion of reactant gas mixture contacts said monolith for no more than about 10 milliseconds when said monolith is employed in a catalyst bed of a millisecond contact time syngas production reactor;
maintaining conversion promoting conditions of temperature, reactant gas composition and pressure and reactant gas/catalyst contact time during said contacting whereby a net partial oxidation reaction is catalyzed by said nickel monolith catalyst.

2. The method of claim 1 wherein said step of contacting comprises contacting a reactant gas mixture comprising said hydrocarbon and O₂ with a catalytically effective amount of a self-supported metallic nickel monolith catalyst having an expanded metal structure sufficiently transparent to pass reactant and/or product gases at a flow rate of at least 2.5 SLPM, said metallic nickel catalyst having been activated by heating in a reducing environment prior to contacting said reactant gas mixture.

3. The method of claim 2 wherein said step of contacting comprises contacting a reactant gas mixture comprising said hydrocarbon and O₂ with a catalytically effective amount of a self-supported metallic nickel monolith catalyst having a nickel metal foam structure sufficiently porous to pass reactant and/or product gases at a flow rate of at least 2.5 SLPM, said metallic nickel catalyst having been activated by heating in a reducing environment prior to contacting said reactant gas mixture.

4. The method of claim 2 wherein said step of contacting comprises contacting a reactant gas mixture comprising said hydrocarbon and O₂ with a catalytically effective amount of a self-supported metallic nickel monolithic catalyst having a metal foam structure sufficiently porous to pass reactant and/or product gases at a flow rate of at least 2.5 SLPM, said catalyst having been activated by heating in a reducing environment prior to contacting said reactant gas mixture.

5. The method of claim 2 wherein said step of contacting comprises contacting a reactant gas mixture comprising said hydrocarbon and a source of oxygen with a catalytically effective amount of a self-supported metallic nickel monolith catalyst having a gauze structure sufficiently porous to pass reactant and/or product gases at a flow rate of at least 2.5 SLPM, said catalyst having been activated by heating in a reducing environment prior to contacting said reactant gas mixture.

6. The method of claim 2 wherein said step of contacting comprises contacting a reactant gas mixture comprising said hydrocarbon and O₂ with a catalytically effective amount of a self-supported metallic nickel monolith catalyst having a perforated foil structure sufficiently porous to pass reactant and/or product gases at a flow rate of at least 2.5 SLPM, said catalyst having been activated by heating in a reducing environment prior to contacting said reactant gas mixture.

7. The method of claim 2 wherein said step of contacting comprises contacting a reactant gas mixture comprising said hydrocarbon and O₂ with a catalytically effective amount of a self-supported metallic nickel monolith catalyst having a spiral structure sufficiently porous to pass reactant and/or product gases at a flow rate of at least 2.5 SLPM, said catalyst having been activated by heating in a reducing environment prior to contacting said reactant gas mixture.

8. The method of claim 1 wherein said step of maintaining said catalyst and said reactant gas mixture at conversion promoting conditions includes maintaining a catalyst temperature of about 600-1,200° C.

9. The method of claim 8 wherein said step of maintaining a temperature comprises maintaining a catalyst temperature of about 700-1,100° C.

10. The method of claim 1 wherein said step of maintaining said catalyst and said reactant gas mixture at conversion promoting conditions during said contacting includes maintaining a reactant gas pressure of about 100-12,500 kPa.

11. The method of claim 10 wherein said step of maintaining said catalyst and said reactant gas mixture at conversion promoting conditions during said contacting includes maintaining a reactant gas pressure of about 130-10,000 kPa.

12. The method of claim 1 further comprising mixing a methane-containing feedstock and an O₂-containing feedstock to provide a reactant gas mixture feedstock having a carbon:oxygen atomic ratio of about 1.25:1 to about 3.3:1.

13. The method of claim 12 wherein said mixing provides a reactant gas mixture feed having a carbon:oxygen ratio of about 1.3:1 to about 2.2:1.

14. The method of claim 12 wherein said mixing provides a reactant gas mixture feed having a carbon: oxygen ratio of about 1. 5: 1 to about 2.2:1.

15. The method of claim 14 wherein said mixing provides a reactant gas mixture feed having a carbon:oxygen ratio of about 2:1.

16. The method of claim 1 wherein said O₂-containing gas farther comprises steam, CO₂, or a combination thereof.

17. The method of claim 1 further comprising mixing a hydrocarbon feedstock and a gas comprising steam and/or CO₂ to provide said reactant gas mixture.

18. The method of claim 1 wherein said C₁-C₅ hydrocarbon comprises at least about 50% methane by volume.

19. The method of claim 18 wherein said C₁-C₅ hydrocarbon comprises at least about 80% methane by volume.

20. The method of claim 19 further comprising preheating said reactant gas mixture.

21. The method of claim 1 further comprising passing said reactant gas mixture over said monolith catalyst at a space velocity of about 20,000-100,000,000 normal liters of gas per kilogram of catalyst per hour (NL/kg/h).

22. The method of claim 21 wherein said step of passing said reactant gas mixture over said monolith catalyst comprises passing said mixture at a space velocity of about 50,000 to about 50,000,000 NL/kg/h.

23. The method of claim 1 further comprising retaining said monolith catalyst in a fixed bed reaction zone.

24. A method of catalytically converting a C₁-C₅ hydrocarbon comprising at least about 50 vol % methane, in the presence of O₂, to a product gas mixture comprising CO and H₂, the method comprising:
mixing a gaseous C₁-C₅ hydrocarbon-containing feedstock and an oxygen-containing feedstock to provide a reactant gas mixture feedstock having a carbon:oxygen ratio of about 1.25:1 to about 3.3:1;
in a millisecond contact time reactor, contacting said reactant gas mixture feedstock with a catalytically effective amount of a self-supported reduced metallic nickel monolith catalyst having sufficient transparency to allow reactant and/or product gases to flow through a catalyst bed of said reactor at such a rate that the contact time for a portion of reactant gas mixture that contacts said monolith catalyst is no more than about 10 milliseconds when said catalyst is used in said reactor;
passing said reactant gas mixture feedstock over said monoliths at such flow rate that the contact time for a portion of reactant gas mixture that contacts said monoliths is not more than about 10 milliseconds;
during said contacting, maintaining said monolith catalyst at a temperature of about 600-1,200° C.;
during said contacting, maintaining said reactant gas mixture at a pressure of about 100-12,500 kPa; and
during said contacting, optionally, adjusting said hydrocarbon and said oxygen concentration in said reactant gas mixture feedstock to a carbon:oxygen ratio is about 1.25:1 to about 3.3: 1, such that the molar ratio of H₂:CO in said product gas mixture is about 2:1.

25. A method of making a self-supported metallic nickel monolith catalyst that is active for catalyzing the partial oxidation of methane in the presence of O₂ to CO and H₂ under partial oxidation promoting conditions of reactant gas composition and pressure, flow rate and temperature, the method comprising:
shaping a bulk nickel metal material such that a metallic nickel monolith is formed having a sufficiently transparent structure to allow reactant and/or product gases to flow through at such a rate that the contact time of a portion of a reactant gas mixture that contacts said shaped bulk nickel material is no more than about 10 milliseconds, when said monolith is used in a catalyst bed of a short contact time reactor;
reducing said metallic nickel to yield an activated nickel monolith catalyst; and,
optionally, maintaining said nickel monolith catalyst in an oxidatively reduced condition until said catalyst is used in a short contact time syngas reactor for catalyzing the production of CO and H₂.

26. The method of claim 25 wherein said reducing step comprises heating said monolith in a reducing environment to yield an activated nickel monolith catalyst.

27. The method of claim 26 wherein said reducing step comprises passing hydrogen gas over said monolith while heating said monolith.

28. The method of claim 27 wherein said reducing step comprises passing hydrogen gas over said monolith at a rate of 100 cc/min while heating said monolith at 800° C. for 4 hours.

29. The method of claim 25 further comprising introducing at least one said metallic nickel monolith into a syngas synthesis reactor prior to said reducing.

30. The method of claim 25 wherein said step of shaping said metallic nickel monolith comprises forming a spiral structure.

31. The method of claim 25 wherein said step of shaping said metallic nickel monolith comprises forming a nickel foam.

32. The method of claim 25 wherein said step of shaping said metallic nickel monolith comprises perforating a nickel foil.

33. The method of claim 25 wherein said step of shaping said metallic nickel monolith comprises cutting a bulk nickel metal blank chosen from the group consisting of nickel gauzes and expanded nickel metal sheets to yield at least one piece.

34. A reduced nickel metal syngas catalyst (Ni⁰) having a three-dimensional form chosen from the group consisting of expanded nickel metal sheets, nickel gauzes, nickel foams, perforated nickel foils and nickel spirals, and having activity for catalyzing the net partial oxidation of methane to synthesis gas such that, at a reactant gas feed composition of about 60% CH₄ and about 30% O₂, the conversion of reactants is about 100% 02 and at least about 95% CH₄ and the selectivity of products is about 95% CO and 70% H₂.

35. The catalyst of claim 34 wherein said three-dimensional form comprises up to 90% open area.

36. The catalyst of claim 34 wherein the mechanical strength of said catalyst is sufficient to withstand an on-stream pressure of at least 100 kPa for at least 6 months.

37. The catalyst of claim 34 wherein the macroporosity of said catalyst is sufficient to pass reactant and/or product gases at a space velocity of at least 20,000 normal liters of gas per kilogram of catalyst per hour (NL/kg/h).
